(19) United States
(12) Patent Application Publication
(10) Pub. No.: US 2010/0234906 A1
(43) Pub. Date: Sep. 16, 2010

(54) SYSTEM AND METHOD FOR CONTROLLING RATE-ADAPTIVE PACING BASED ON A CARDIAC FORCE-FREQUENCY RELATION DETECTED BY AN IMPLANTABLE MEDICAL DEVICE

(75) Inventor: Steve Koh, South Pasadena, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/405,090

(22) Filed: Mar. 16, 2009

(51) Int. Cl. A61N 1/365; A61M 1/00

(52) U.S. Cl. 607/3; 607/18

(57) ABSTRACT

Techniques are provided for use in controlling rate-adaptive pacing within implantable medical devices such as pacemakers or implantable cardioverter-defibrillators (ICDs). In one example, a force-frequency relationship is determined for the heart of the patient, which is representative of the relationship between cardiac stimulation frequency and myocardial contractile force. To this end, various parameters are detected for use as surrogates for contractile force, including selected systolic pressure parameters and cardiogenic impedance parameters. Rate-adaptive pacing is then controlled based on the detected force-frequency relationship to, for example, deactivate rate-adaptive pacing if the slope and/or abscissa of the force-frequency relationship indicates significant contractility dysfunction within the patient. In other examples, rather than deactivating rate-adaptive pacing, control parameters are adjusted to render the rate-adaptive pacing less aggressive. In still other examples, trends in the slope and/or abscissa of the force-frequency relationship are monitored to detect contractility dysfunction and/or heart failure and titrate medications accordingly.

### FIELD OF THE INVENTION

The invention generally relates to implantable medical devices, such as pacemakers or implantable cardioverter-defibrillators (ICDs), and in particular to techniques for controlling rate-adaptive pacing within patients in which such devices are implanted.

### BACKGROUND OF THE INVENTION

A pacemaker is an implantable medical device that recognizes various arrhythmias such as an abnormally and delivers electrical pacing pulses to the heart in an effort to remedy the arrhythmias. An ICD is an implantable device that additionally or alternatively recognizes ventricular tachycardia (VT) and/or ventricular fibrillation (VF) and delivers electrical shocks or other therapies to terminate these tachyarrhythmias.

Many state-of-the-art pacemakers and ICDs are equipped to perform rate-adaptive pacing. With rate-adaptive pacing (sometimes also referred to as rate-responsive pacing or rate-variable pacing), the device automatically changes the rate at which pacing pulses are delivered to the heart of the patient so as to meet changing metabolic demands. Rate-adaptive pacing is typically used with patients whose heart rates do not naturally increase in response to exercise (i.e. chronotropic incompetence). The rate-adaptive device senses a physiologic parameter indicative of exercise and provides a corresponding increase in the pacing rate to meet the metabolic demands of that exercise.

Problems, however, can arise within patients with heart failure or with other medical conditions affecting myocardial contractility. Heart failure is a debilitating disease in which abnormal function of the heart leads in the direction of inadequate blood flow to fulfill the needs of the tissues and organs of the body. The heart can lose propulsive power if the cardiac muscle loses its capacity to contract and/or stretch (i.e. there is at least some dysfunction in contractility and/or disentsibility.) Often, the ventricles do not adequately eject or fill with blood between heartbeats and the valves regulating blood flow become leaky, allowing regurgitation or back-flow of blood. The impairment of arterial circulation deprives vital organs of oxygen and nutrients.

Hence, with heart failure or other conditions, the contractility of the myocardium can be impaired, i.e. there is some degree of contractility dysfunction. Within some patients with contractility dysfunction, an attempt to increase the pacing rate in response to exercise can be problematic, since the heart will not be able to adequately respond to the increased rate given the dysfunction. Indeed, in some cases, a myocardial infarction can result.

Accordingly, it would be desirable to provide techniques for detecting contractility dysfunction within patients and for adjusting or deactivating rate-adaptive pacing. It is to this end that the invention is primarily directed. Additionally, it is desirable to provide new and improved techniques for detecting and tracking heart failure based on contractility dysfunction and other aspects of the invention are directed to this end.

### SUMMARY OF THE INVENTION

In accordance with exemplary embodiments of the invention, techniques are provided for use in controlling rate-adaptive pacing within implantable medical devices. In one example, a force-frequency relationship is determined for the heart of the patient, which is representative of the relationship between cardiac stimulation frequency and contractile force. Then, rate-adaptive pacing is controlled based on the force-frequency relationship to for example, deactivate rate-adaptive pacing if the force-frequency relationship indicates contractility dysfunction within the patient.

Briefly, the force-frequency relation is a relationship between the force of contraction of the myocardium of the heart and the rate at which the heart beats (either intrinsic heartbeats or beats triggered by pacing pulses.) Within a healthy heart, the force of contraction increases significantly with increasing heart rate in accordance with the so-called Treppe phenomenon or the Bowditch effect. However, in a failing myocardium, the normal increase in contractile force with increasing heart rate can be diminished considerably. Indeed, within some patients, the contractile force can decrease with increasing heart rate. Within patients with contractility dysfunction, rate-adaptive pacing may be inappropriate. These patients are referred to herein as exhibiting a lack of responsiveness to rate-adaptive pacing.

Accordingly, within at least some embodiments of the invention, the implantable medical device examines the force-frequency relation for the patient to detect an indication of a lack of responsiveness to rate-adaptive pacing and then adjusts or controls rate-adaptive pacing within the patient. For example, a programmed “maximum rate-adaptive pacing rate” may be lowered in response to an indication of a lack of responsiveness to rate-adaptive pacing. As another example, a programmed “reaction time” associated with rate-adaptive pacing may be decreased. Recovery rates may also be adjusted. In still another example, rate-adaptive pacing is simply deactivated or suspended. In some embodiments, any adjustments to rate-adaptive pacing depend upon the current exercise level of the patient. That is, rate-adaptive pacing may be performed so long as the exercise level is relatively low, but is deactivated if the exercise rate becomes too high.

To determine the force-frequency relation within the patient, in one example, the implantable device detects values representative of contractile force within the left ventricle (LV) of the patient over a range of heart rates while recording both the contractile force and the corresponding heart rate. This set of paired values represents the force-frequency relationship for the patient. From the paired values, the pacer/ICD can determine the slope and/or abscissa of the force-frequency relationship for use in controlling rate-adaptive pacing or other functions.

Within patients in which rate-adaptive pacing is enabled, assuming the patient is sufficiently active, rate-adaptive pacing will occasionally be triggered in response to patient exercise. While the pacing rate is automatically increased by the rate-adaptive pacing system during the periods of exercise, the device can collect contractile force/pacing rate data from which the force-frequency relationship can be determined. In other words, no special pacing session is required to obtain this data. Within active patients in which rate-adaptive pacing is not enabled, the heart rate can be passively monitored as it naturally varies with patient activity over a range of heart rates to allow the device to obtain contractility force values. Preferably, contractility values are obtained over a range of rates from the resting rate of the patient to at least 100 beats per minute (bpm). As an option, for any patients who might not be sufficiently active to produce significant heart rate increases (either naturally or via rate-adaptive pacing in response to exercise), the heart of the patient can be paced at different rates over the preferred range of heart rates. This is particularly useful within patients who are generally sedentary.

To determine contractile force, any of a variety of surrogate parameters may be detected by the implanted device. For example, the device can detect: a time rate of change of cardiogenic impedance (dZ/dt); a time rate of change of cardiac pressure (dP/dt); a peak systolic pressure (P); or a peak-to-peak amplitude of cardiogenic impedance (Z). Cardiogenic impedance may be detected by delivering suitable impedance detection pulses to the heart of the patient via pacing/sensing electrodes (while filtering out variations in detected impedance waveforms due respiration or other non-cardiogenic factors.) Cardiac pressure may be detected using a suitable pressure transducer implanted within the heart, such as a left atrial pressure (LAP) sensor. Lead-based photo-plethysmography (PPG) sensors can also be used to determine suitable pressure values for use as a surrogate for contractile force.

In an illustrative embodiment, the surrogate values representative of contractile force are recorded along with the corresponding heart rate over a predetermined range of heart rates to represent the force-frequency relationship. Curve-fitting is then employed to determine the slope and abscissa of the force-frequency relationship. The current force-frequency slope is then compared against a previously determined baseline slope for the patient to detect any significant changes in the slope. In general, a significant decrease in the slope is indicative of a lack of responsiveness to rate-adaptive pacing. In one example, if amount of the decrease in slope exceeds a percentage threshold, rate-adaptive pacing is deactivated. By comparing the current slope of the force-frequency curve against a baseline slope for the patient, the surrogate values for contractile force need not be calibrated to contractile force units. That is, the device need not determine the actual contractile force of the myocardium of the patient from the surrogate values. Rather, it is sufficient to detect a significant change in the slope derived from the surrogate values within the patient. Additionally, or alternatively, the device compares the abscissa of the force-frequency relationship against a previously determined baseline abscissa for the patient to detect any significant changes in the abscissa.

Additionally, a significant decrease in the slope and/or abscissa of the force-frequency relationship (relative to baseline values) can be associated with increasing contractility dysfunction and with heart failure progression. Warning signals can be generated accordingly. Again, suitable thresholds can be used. Diagnostic data is preferably recorded indicative of the force-frequency relationship. If the implanted device is equipped with a drug pump, suitable medications can be automatically administered (or their dosages titrated) in response to a significant decrease in force-frequency slope and/or abscissa. In one example, verapamil is delivered to the patient to reduce ventricular-vascular stiffening so as to increase contractile force.

System and method examples of the invention are described herein below.

### BRIEF DESCRIPTION OF THE DRAWINGS

The above and further features, advantages and benefits of the invention will be apparent upon consideration of the present description taken in conjunction with the accompanying drawings, in which:

FIG. 1 illustrates pertinent components of an implantable medical system having a pacemaker or ICD capable of determining, monitoring and exploiting the force-frequency relationship of the heart of a patient;

FIG. 2 is a flowchart providing a broad overview of a technique performed by the system of FIG. 1 for controlling rate-adaptive pacing based on the force-frequency relationship and/or for detecting contractility dysfunction, heart failure, etc.;

FIG. 3 illustrates an exemplary technique for determining the force-frequency relationship within a patient for use with the general technique of FIG. 2, which utilizes various pressure and impedance parameters as surrogates for contractile force;

FIG. 4 is a graph illustrating an exemplary force-frequency relationship of the type determined by the technique of FIG. 3, as well as its slope and abscissa;

FIG. 5 is a block diagram summarizing system components that can be used with the technique of FIG. 2 to determine the force-frequency relationship;

FIG. 6 illustrates an exemplary technique for controlling rate-adaptive pacing for use with the general technique of FIG. 2, which also provides for monitoring of contractility dysfunction and heart failure as well as titration of medication;

FIG. 7 includes graphs illustrating changes in the slope of the force-frequency relationship over time, which may be exploited by the technique of FIG. 6 to control rate-adaptive pacing and to titrate medication;

FIG. 8 is a simplified, partly cutaway view, illustrating the pacer/ICD of FIG. 1 along with at set of leads implanted into the heart of the patient; and

FIG. 9 is a functional block diagram of the pacer/ICD of FIG. 8, illustrating basic circuit elements that provide cardioversion, defibrillation and/or pacing stimulation in the heart and particularly illustrating components for determining, monitoring and exploiting the force-frequency relationship within the heart of a patient using the techniques of FIGS. 2-7.

### DETAILED DESCRIPTION OF THE PREFERRED EMBODIMENTS

The following description includes the best mode presently contemplated for practicing the invention. This description is not to be taken in a limiting sense but is made merely to describe general principles of the invention. The scope of the invention should be ascertained with reference to the issued claims. In the description of the invention that follows, like numerals or reference designators will be used to refer to like parts or elements throughout.

### Overview of Implantable System

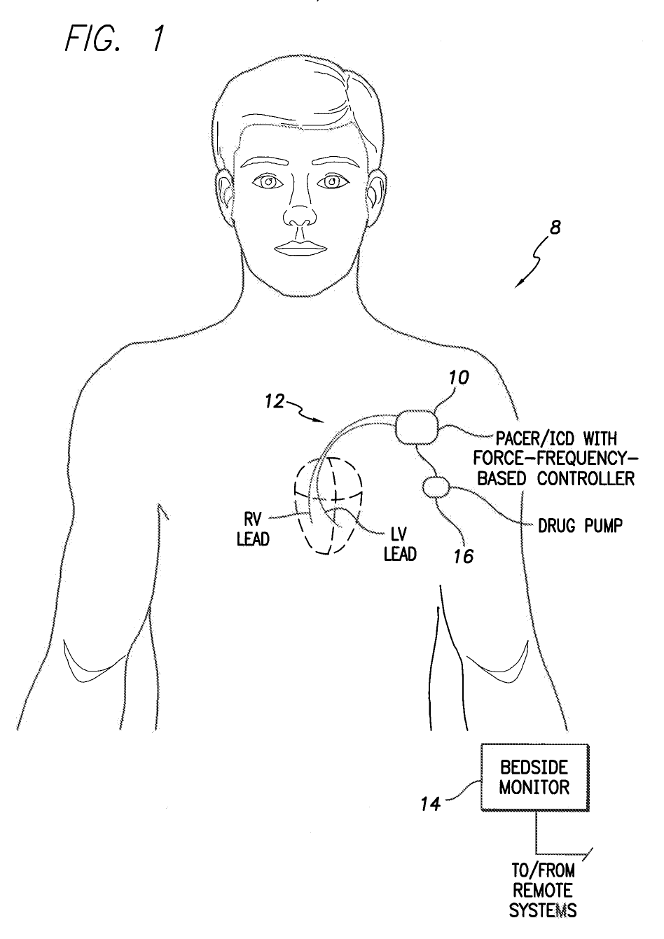

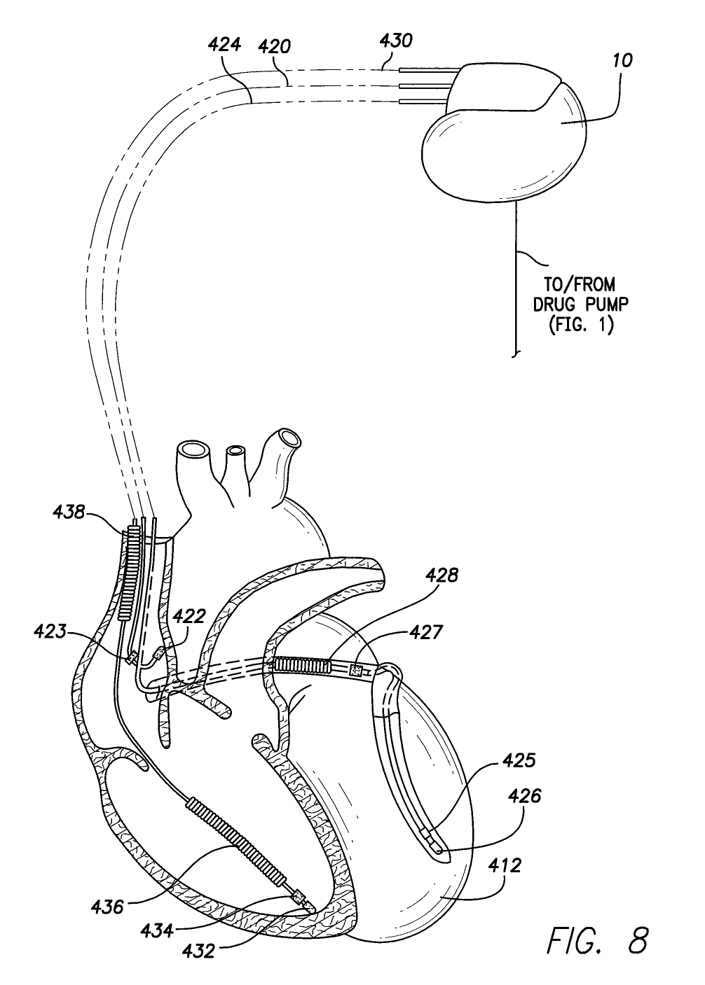

FIG. 1 illustrates an implantable medical system 8 capable of determining, monitoring and exploiting the force-frequency relationship within the heart of a patient and, in particular, for controlling rate-adaptive pacing based on the force-frequency relationship. To this end, medical system 8 includes a pacer/ICD 10 or other cardiac rhythm management device capable of detecting one or more parameters representative of myocardial contractile force via cardiac sensing/pacing leads 12 implanted within the heart of the patient (which may be equipped with pressure sensors, PPG sensors, or the like, not shown.) In FIG. 1, two exemplary leads are shown—an RV lead and an LV lead, in stylized form. A more complete set of leads is illustrated in FIG. 8.

The parameters representative of contractile force detected using the leads may be surrogates for myocardial contractility derived from cardiogenic impedance signals, cardiac pressure signals, PPG signals, etc. (to be discussed in greater detail below.) The surrogates for contractile force are paired with corresponding heart rate values (either paced or sensed) to generate the force-frequency relationship for the patient for use in controlling rate-adaptive pacing or, in some examples, evaluating contractility dysfunction and detecting and tracking heart failure.

Warning signals may be generated to warn of contractility dysfunction, heart failure or other issues using an internal warning device within the pacer/ICD, a bedside monitor 14, or a hand-held personal advisory module (PAM), not separately shown. The internal warning device (not shown in FIG. 1) may be a vibrating device or a “tickle” voltage device that, in either case, provides perceptible stimulation to the patient to alert the patient. The bedside monitor or PAM may provide audible or visual alarm signals to alert the patient, as well as any appropriate textual or graphic displays. In some examples, the implantable system may be equipped with a drug pump 16 capable of the delivering medications to patient tissues in an attempt to address contractility dysfunction or other issues. Implantable drug pumps for use in dispensing medications are discussed in U.S. Pat. No. 5,328,460 to Lord, et al., entitled “Implantable Medication Infusion Pump Including Self-Contained Acoustic Fault Detection Apparatus.” This patent also discusses implantable “tickle” warning devices that may be used to deliver warning signals.

Diagnostic information pertaining to the force-frequency relationship, rate-adaptive pacing, contractility dysfunction, heart failure or other conditions may be stored within the pacer/ICD for transmission to the PAM, bedside monitor or to an external programmer (not shown in FIG. 1) for review by a clinician. The clinician then prescribes any appropriate drug therapies. The clinician may also adjust the operation of the pacer/ICD to activate, deactivate or otherwise control any therapies that are automatically applied. In addition, the bedside monitor may be directly networked with a centralized computing system for immediately notifying the clinician or other caregiver of any concerns. The centralized system may include such systems as the HouseCall™ system or the Merlin@home/Merlin.Net systems of St. Jude Medical. A system incorporating bedside monitoring units connected to a centralized external programmer system is described in U.S. Pat. No. 6,622,045 to Snell et al., “System and Method for Remote Programming of Implantable Cardiac Stimulation Devices.”

The pacer/ICD may also be programmed to activate or control any pacing therapies that might be appropriate in response to contractility dysfunction, heart failure or other medical conditions, such as delivering CRT in response to the detection of heart failure. Additionally, the pacer/ICD performs a wide variety of pacing and/or defibrillation functions such as delivering pacing is response to arrhythmias or generating and delivering shocks in response to ventricular fibrillation.

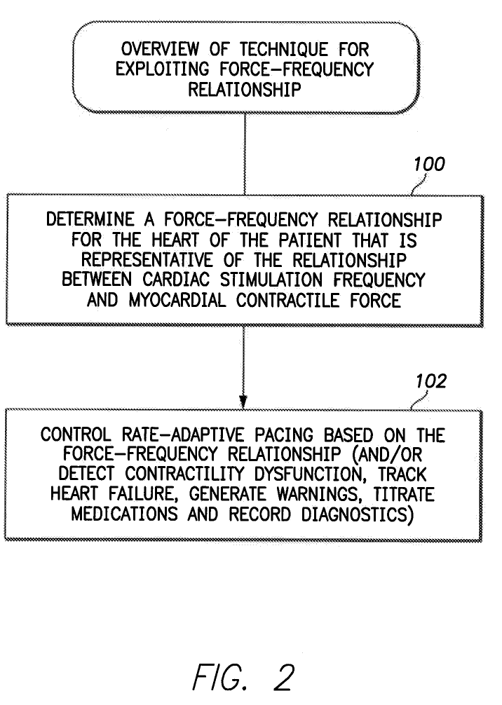

FIG. 2 broadly summarizes the general technique for exploiting the force-frequency relationship, which may be performed by the implantable system of FIG. 1 or other suitably equipped systems. Beginning at step 100, the pacer/ICD determines a force-frequency relationship for the heart of the patient that is representative of the relationship between cardiac stimulation frequency and myocardial contractile force. At step 102, the pacer/ICD then controls rate-adaptive pacing based on the force-frequency relationship (and/or detects contractility dysfunction, tracks heart failure, generates warnings, titrates medications and records diagnostics, etc.)

Hence, FIGS. 1 and 2 provide an overview of an implantable system and method capable of determining and monitoring the force-frequency relationship of the patient and further capable of controlling rate-adaptive pacing, detecting heart failure, titrating medications, delivering appropriate warnings, if needed, etc. Embodiments may be implemented that do not necessarily perform all of these functions. For example, embodiments may be implemented that provide only for controlling rate-adaptive pacing based on the force-frequency relationship without necessarily detecting or tracking heart failure. In addition, systems provided in accordance with the invention need not include all the components shown in FIG. 1 such as the bedside monitor or the implantable drug pump. No attempt is made herein to describe all possible combinations of components that may be provided in accordance with the general principles of the invention. Also, note that, the particular shape, size and locations of the implanted components shown in FIG. 1 are merely illustrative and may not necessarily correspond to actual implant locations. In particular, preferred implant locations for the leads are more precisely illustrated in FIG. 8.

### Exemplary Force-Frequency-Based Monitoring Techniques

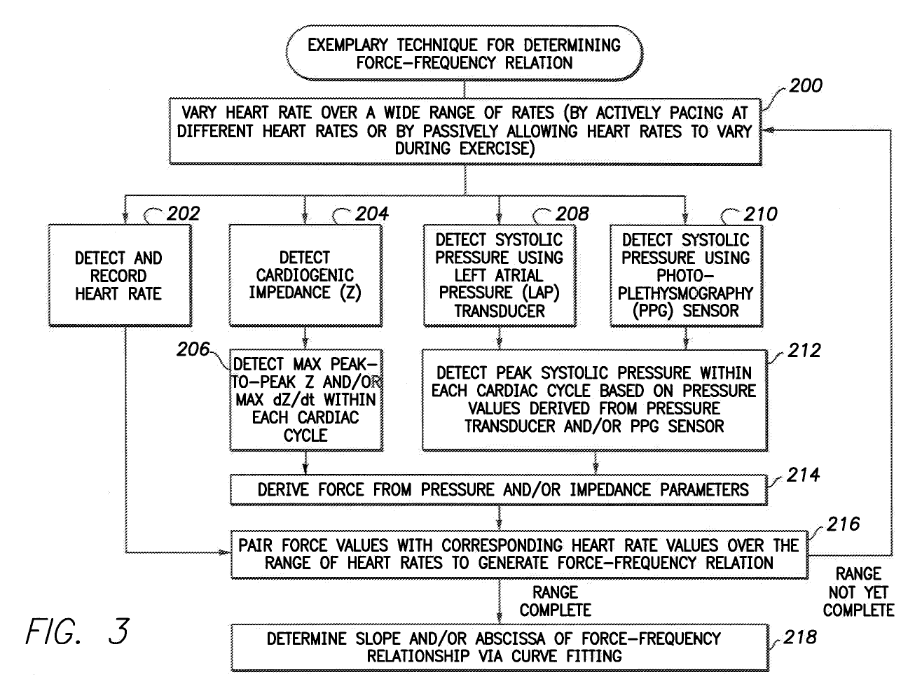

FIGS. 3-5 illustrate an exemplary technique for determining the force-frequency relationship within a patient in a manner that does not require calibration of force values. Beginning at step 200 of FIG. 3, patient heart rate is varied over a range of rates. This may be achieved by actively pacing the heart at different rates or by allowing rates to vary passively due to exercise. Active pacing may be needed for patients who are generally sedentary. In such patients, the device can be programmed to periodically vary the patient's heart rate through a predetermined range of rates extending, for example, from the resting rate of the patient up to 100 bpm or more and then back to the resting rate. Data is collected while the rate is being increased and then decreased. This may be done, e.g., once per day. (Preferably, the patient is advised in advance that this will occur so that the patient will not be unduly alarmed by the changing heart rate.)

For patients who are more active, the pacing rate will vary in accordance with patient exercise or activity over the course of day. In one example, if rate-adaptive pacing is currently enabled within an active patient, then whenever the patient's level of exercise increases significantly, the pacer/ICD will automatically increase the pacing rate to meet the metabolic demands of the exercise. Data is collected while the heart rate is being increased and later decreased. If rate-adaptive pacing is not enabled, the pacer/ICD may be programmed to passively track the patient's heart rate to collect data in various heart rate ranges. That is, for active patients, at some point during the day the heart rate will likely increase to 100 bpm or more due to exercise and, at that time the pacer/ICD can collect the needed data. If the heart rate does not vary throughout the entire desired range over the course of a given day, active pacing may then be employed to pace the heart throughout the range to collect the data.

In any case, while the heart rate is actively or passively varied over the range of rates, the pacer/ICD detects and records the current rate at step 202 while concurrently collecting data from which contractile force can be derived. In this example, several different surrogates for contractile force are used.

At step 204, the pacer/ICD detects cardiogenic impedance (Z) by applying impedance detection pulses between, e.g., LV and RV pacing electrodes. Suitable filters may be employed to isolate the cardiogenic portion of the signal and exclude lower frequency components, such as respiratory components. A particularly effective tri-phasic impedance detection pulse for use in detecting impedance is described in U.S. patent application Ser. No. 11/558,194 of Panescu et al., filed Nov. 9, 2006, entitled “Closed-Loop Adaptive Adjustment of Pacing Therapy based on Cardiogenic Impedance Signals Detected by an Implantable Medical Device.” However, other impedance detection pulses or waveforms may instead be exploited.

Then, at step 206, for each of a set of cardiac cycles (i.e. heartbeats), the pacer/ICD detects the maximum peak-to-peak change in Z during the cardiac cycle. By “maximum peak-to-peak,” it is meant that the pacer/ICD detects the maximum and minimum amplitude of the Z signal within a given cardiac cycle and records the magnitude of the difference between the two values. The max peak-to-peak value is indicative of blood flow through the aortic arch and, in general, the stronger the contractile force of the heart, the greater the max peak-to-peak Z value. As such, max peak-to-peak Z values can be used as a surrogate for contractile force. Preferably, these values are averaged over a set of cardiac cycles at each heart rate of interest. Additionally, or alternatively, the pacer/ICD determines the maximum dZ/dt value for each of a set of cardiac cycles and averages the values together. Here, dZ/dt represents the time rate of change of the signal, i.e. its slope. The maximum dZ/dt value is the maximum slope of the signal. In general, the stronger the contractile force of the heart, the larger the max dZ/dt value. As such, max dZ/dt values can also be used as a surrogate for contractile force.

At step 208, for each of set of cardiac cycles, the pacer/ICD detects pressure using a lead-based pressure transducer, such as an LAP sensor (if the implantable system is so equipped.) LAP sensors described, for example, in U.S. patent application Ser. No. 11/927,026, filed Oct. 29, 2007, entitled “Systems and Methods for Exploiting Venous Blood Oxygen Saturation in Combination with Hematocrit or other Sensor Parameters for use with an Implantable Medical Device.” Concurrently, at step 210, the pacer/ICD, uses a lead-based PPG sensor (if so equipped) to detect systolic pressure. PPG sensors are also discussed in U.S. patent application Ser. No. 11/927,026. Then, at step 212, the pacer/ICD detects peak systolic pressure within each cardiac cycle based on pressure values derived from pressure transducer and/or PPG sensor. In general, the greater the peak systolic pressure, the stronger the contractile force. As such, peak systolic pressure can also be used as a surrogate for contractile force.

At step 214, the pacer/ICD then derives contractile force from the various pressure and impedance values. As noted, these values are used as surrogates for contractile force and so it is not necessary to perform any numerical conversion of the pressure and impedance values into actual force values. As such, no calibration is required to convert the pressure and impedance values into force values for each individual patient. (Though, if such calibration values are available, and if the pacer/ICD is so equipped, it certainly can be programmed to convert the pressure and impedance values into actual force values.)

After storing the latest pairs of heart rate/contractile force surrogate values, if data pairs have not yet been collected throughout the entire range of values, processing returns to step 200 to further vary the heart rate to collect more data. In one example, the range of heart rates is divided into sub-ranges and at least one pair of heart rate/contractile force values is stored for each sub-range. For example, within an overall range of 40 bpm-100 bpm, sub-ranges may be defined as follows: 40-45 bpm, 46-50 bpm, 51-55 bpm, 56-60 bpm, etc. If data has been collected throughout the overall range of heart rates, then processing continues to step 218 where the pacer/ICD determines the slope and/or abscissa (i.e. the force at a given heart rate such as at a base pacing rate) of the force-frequency relationship via curve fitting.

Note that, if several different types of surrogate values have been detected (e.g., max peak-to-peak Z and max dZ/dt values are detected along with both LAP and PPG pressure values), a separate force-frequency slope value may be independently calculated for each separate parameter. Then, the separate force-frequency slope values are averaged together to yield a single slope value for subsequent use in controlling rate-adaptive pacing, detecting contractility dysfunction, etc. Similarly, separate abscissa values may be determined based on the different surrogate parameters. In other implementations, though, it may be appropriate to combine the various surrogate parameters together into a single surrogate metric value, from which a single force-frequency slope and/or abscissa value is calculated. Note also that max peak-to-peak Z is similar to max systolic volume (SV) measured along RA ring-Can vector, which can be interpreted as contractility.

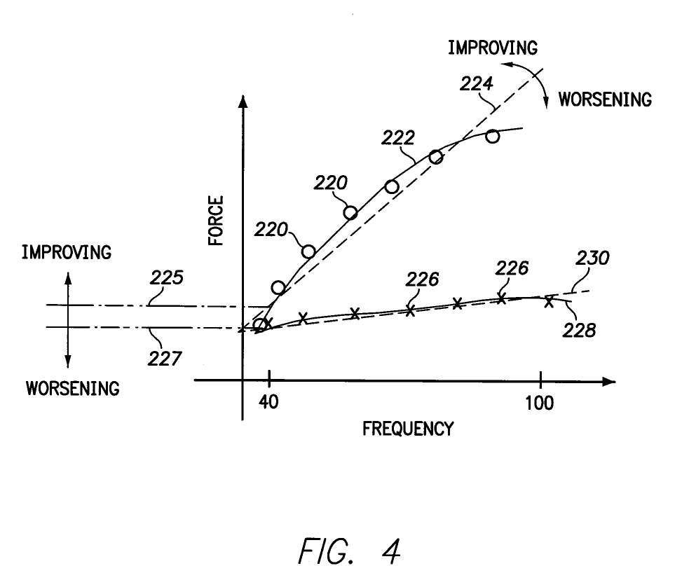

FIG. 4 illustrates exemplary force-frequency data points and the force-frequency relationships represented thereby. Note that “frequency” refers to the heart rate. Force is shown in arbitrary units as the values used (max dZ/dt values, in this case) are surrogates for contractile force, rather than actual calibrated force values. A first set of data points 220 (shown as small circles) corresponds to the force-frequency curve 222 of a nonfailing heart. A straight line 224 is fitted to the data points to represent the slope of the force-frequency curve. As can be seen, the slope is fairly steep, indicating a significant increase in contractile force with increasing pacing heart rate in accordance with the Treppe phenomenon. A second set of data points 226 (shown as small x's) corresponds to the force-frequency curve 228 of a failing heart. A straight line 230 is fitted to the data points to represent its slope. As can be seen the slope is fairly flat, indicative of minimal increase in contractile force with increasing pacing heart rate. The minimal increase in contractile force with increasing pacing heart rate is indicative of contractility dysfunction sometimes found in heart failure patients. [Note that the data shown in FIG. 4 is hypothetical data provided merely to illustrate the invention and should not be construed as representing actual clinically-detected data from human patients.]

FIG. 4 also shows the abscissa of the force-frequency curve, which represents the force at a given heart rate, such as at the base pacing rate. Horizontal line 225 identifies the force at a base rate of 40 bpm for a nonfailing heart. Horizontal line 227 is the force at a base rate of 40 bpm for a failing heart.) The abscissa value is typically higher for a healthy heart as opposed to a failing heart.

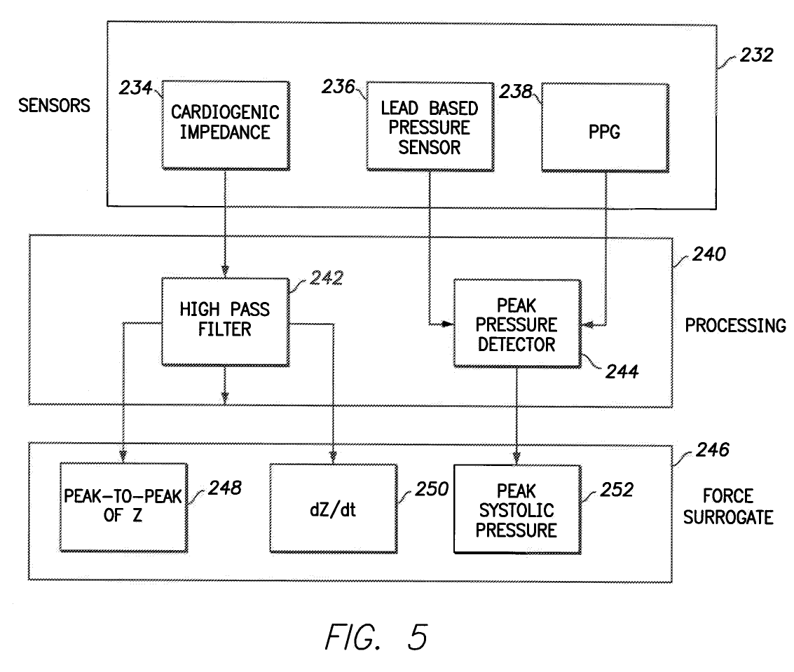

FIG. 5 illustrates exemplary system components for use in collecting the data for use by the technique of FIG. 4 to determine the force-frequency relationship. Briefly, a sensor block 232 includes a cardiogenic impedance detector 234, a lead-based pressure sensor 236 and a PPG sensor 238. Output from the sensors is fed into a processing block 240. More specifically, cardiogenic impedance data is fed into a high-pass filter 242, which filters noise from the signal. (Although not shown, to obtain the initial cardiogenic impedance signal, a low-pass filter may be used to filter out respiratory components or other low frequency signal components.) Data from the pressure sensor and the PPG sensor are fed into a peak pressure detector, which detects the peak pressure within a given cardiac cycle. Output from the processing block is then fed into a force surrogate block 246, which determines the various surrogate parameters discussed above. In particular, a peak-to-peak Z block 248 determines the max peak-to-peak Z value within a given cardiac cycle. A dZ/dt block 250 determines the max dZ/dt value within a given cardiac cycle. A peak systolic pressure block 252 determines the peak systolic pressure within a given cardiac cycle.

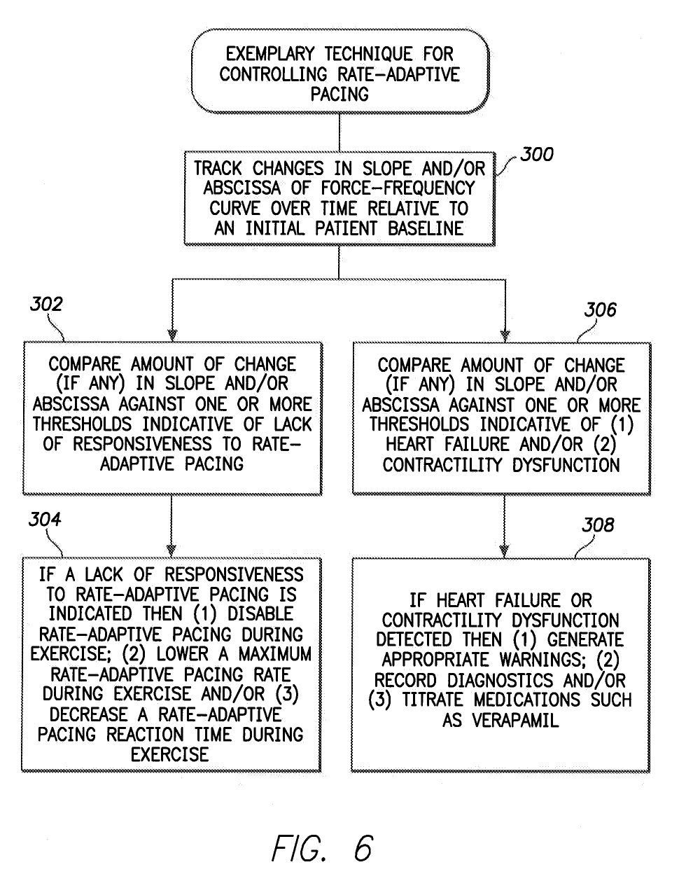

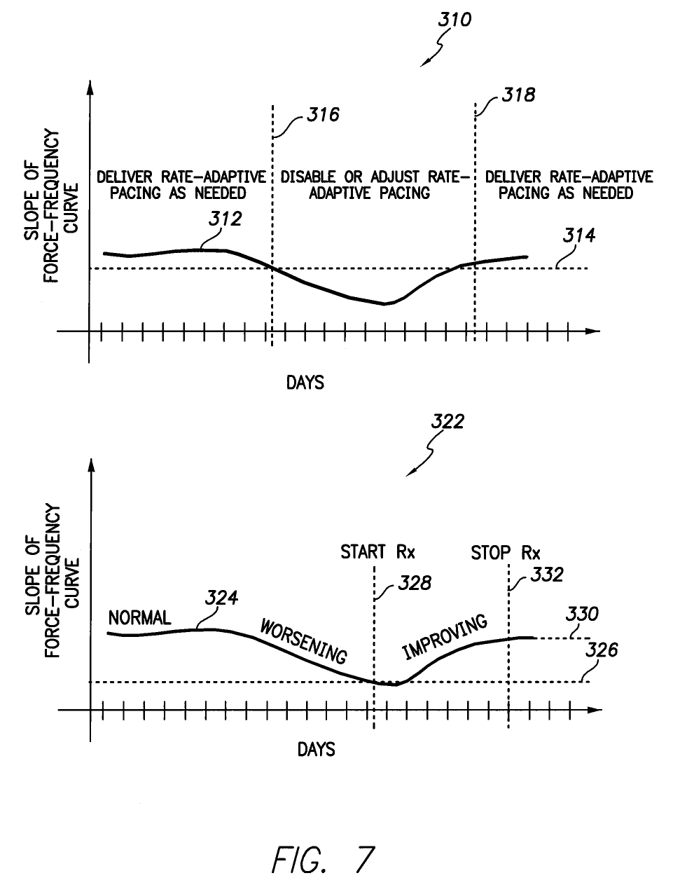

Turning now to FIGS. 6-7, exemplary techniques for detecting and exploiting trends in the slope and/or abscissa of the force-frequency relationship will now be described. Beginning at step 300 of FIG. 6, the pacer/ICD tracks changes in the slope and/or abscissa of the force-frequency curve over time relative to an initial patient baseline. Changes in slope and/or abscissa can be quantified, e.g., as a percentage change relative to baseline values. The baseline value may be determined initially for the patient, for example, during a follow-up programming session with the clinician following device implant. By comparing newly detected slope and/or abscissa values against baseline values, changes from baseline can be easily quantified and detected within the patient. Typically, a new slope and/or abscissa value is detected and recorded each day. Trends over time are tracked. In general (as indicated in FIG. 5), a decrease in slope/abscissa is indicative of the progression or worsening of myocardial contractile force (and also in any conditions that might degrade that force, such as cardiomyopathy and heart failure.) An increase in the slope/abscissa is indicative of improvement in contractile force (and also in any related conditions such as cardiomyopathy and heart failure.)

At step 302, the pacer/ICD compares the amount of change (if any) in the slope and/or abscissa against one or more thresholds indicative of lack of responsiveness to rate-adaptive pacing. For example, a 25% decrease in slope relative to the baseline might be used to detect lack of responsiveness. A different percentage may be used for the abscissa. If a lack of responsiveness to rate-adaptive pacing is indicated, then, at step 304, the pacer/ICD: (1) disables rate-adaptive pacing during exercise; (2) lowers a maximum rate-adaptive pacing rate during exercise and/or (3) decreases a rate-adaptive pacing reaction time during exercise. Recovery rates may also be adjusted. As noted above, rate-adaptive pacing may be inappropriate within patients suffering contractility dysfunction (as indicated by a poor force-frequency slope and/or abscissa value.) Accordingly, it is best to either deactivate rate-adaptive pacing within such patients or, at least make the pacing less aggressive by lowering the maximum rate-adaptive pacing rate or decreasing the rate-adaptive pacing reaction time.

The specific actions performed by the pacer/ICD will depend on device programming, as selected by the clinician. In some implementations, in response to an initial decrease in the force-frequency slope and/or abscissa values, the device first makes rate-adaptive pacing less aggressive by adjusting its parameters. If the force-frequency values continue to decrease, then rate-adaptive pacing may be deactivated within the patient. Suitable threshold values for use in distinguishing among these different states may be determined via otherwise conventional clinical testing. Also, note that in the illustrated example rate-adaptive pacing is only adjusted or disabled while the patient is exercising or otherwise active (as detected by an activity sensor.) Rate-adaptive pacing is not adjusted or disabled at other times since rate adjustments, if any, will be modest. In other examples, though, rate-adaptive pacing may be adjusted or disabled at all times (in response to a poor force-frequency slope and/or abscissa value.)

Additionally, or alternatively, the pacer/ICD may be equipped detect heart failure, titrate medications, etc., in response to changes in the force-frequency values. For example, at step 306, the pacer/ICD compares the amount of change (if any) in force-frequency slope and/or abscissa against one or more thresholds indicative of (1) heart failure and/or (2) contractility dysfunction. Suitable threshold values for distinguishing among these different conditions may be determined in advance via otherwise conventional clinical testing. At step 308, if heart failure or contractility dysfunction is detected, then the pacer/ICD (1) generates appropriate warnings, (2) records diagnostics and/or (3) titrates medications, such as verapamil. If the medications are delivered via an implantable drug pump, such titration can be automatic. In other patients, suitable instructions are transmitted to the bedside monitor or PAM instructing the patient (or caregiver) to adjust dosages.

FIG. 7 illustrates exemplary trends in force-frequency slope and the actions in response thereto. Again, force is shown in arbitrary units as the values shown are surrogates for contractile force, rather than actual calibrated force values. A first graph 310 illustrates changes in time in the force-frequency slope 312. During an initial period of time, rate-adaptive pacing is delivered as needed in accordance with otherwise conventional rate-adaptive pacing techniques. As can be seen, though, the slope begins to decrease. Once the slope falls below a threshold 314 at time 316, rate-adaptive pacing is deactivated or adjusted to be less aggressive. Eventually, the slope begins to improve (e.g., due to delivery of appropriate medications.) Once the slope exceeds the threshold, at time 318, conventional rate-adaptive pacing resumes.

A second graph 322 also illustrates changes in the force-frequency slope 324 over time. As can be seen, the slope begins to decrease, indicating worsening heart failure and/or contractility dysfunction. Once the slope falls below a first threshold 326 at time 328, a regime of medications is initiated to improve contractility. Eventually, the slope begins to improve. Once the slope exceeds a second threshold 330, at time 332, the medications are suspended.

Although not shown in FIG. 7, similar trends may be tracked in the abscissa value. In some implementations, both the slope and abscissa are combined into a suitable metric value which is then tracked over time.

Insofar as detecting contractility dysfunction and/or heart failure is concerned, the force-frequency-based techniques of the invention can be supplemented with (or corroborated by) other detection techniques. Alternative techniques for detecting contractility values are described in: U.S. Pat. No. 5,800,467 to Park et al., entitled “Cardio-Synchronous Impedance Measurement System for an Implantable Stimulation Device.” Alternative techniques for detecting or tracking heart failure are set forth in the following patents: U.S. Pat. No. 6,748,261, entitled “Implantable Cardiac Stimulation Device for and Method of Monitoring Progression or Regression of Heart Disease by Monitoring Interchamber Conduction Delays”; U.S. Pat. No. 6,741,885, entitled “Implantable Cardiac Device for Managing the Progression of Heart Disease and Method”; U.S. Pat. No. 6,643,548, entitled “Implantable Cardiac Stimulation Device for Monitoring Heart Sounds to Detect Progression and Regression of Heart Disease and Method Thereof”; U.S. Pat. No. 6,572,557, entitled “System and Method for Monitoring Progression of Cardiac Disease State using Physiologic Sensors”; and U.S. Pat. No. 6,480,733, entitled “Method for Monitoring Heart Failure” and U.S. Pat. No. 6,438,408, entitled “Implantable Medical Device For Monitoring Congestive Heart Failure.” See, also, U.S. Patent Application 2007/0043299, filed Feb. 22, 2007, entitled “Tracking Progression of Congestive Heart Failure via a Force-Frequency Relationship.”

What have been described are various techniques for determining and exploiting the force-frequency relationship within the heart of a patient. For the sake of completeness, a detailed description of an exemplary pacer/ICD for performing these techniques will now be provided. However, principles of invention may be implemented within other pacer/ICD implementations or within other implantable devices such as stand-alone monitoring devices, CRT devices or CRT-D devices. (A CRT-D is a cardiac resynchronization therapy device with defibrillation capability.) Furthermore, although examples described herein involve processing of force-frequency data by the implanted device itself, some operations may be performed using an external device, such as a bedside monitor, device programmer, computer server or other external system. For example, recorded heart rate and contractile force surrogate parameters may be transmitted to the external device, which processes the data to evaluate the force-frequency relationship. Processing by the implanted device itself is preferred as that allows the device to easily control rate-adaptive pacing, as well as to detect the onset of contractility dysfunction and to issue prompt warnings.

### Exemplary Pacer/ICD

FIG. 8 provides a simplified block diagram of the pacer/ICD, which is a dual-chamber stimulation device capable of treating both fast and slow arrhythmias with stimulation therapy, including cardioversion, defibrillation, and pacing stimulation, as well as capable of performing the force-frequency functions described above. To provide atrial chamber pacing stimulation and sensing, pacer/ICD 10 is shown in electrical communication with a heart 412 by way of a left atrial lead 420 having an atrial tip electrode 422 and an atrial ring electrode 423 implanted in the atrial appendage. Pacer/ICD 10 is also in electrical communication with the heart by way of a right ventricular lead 430 having, in this embodiment, a ventricular tip electrode 432, a right ventricular ring electrode 434, a right ventricular (RV) coil electrode 436, and a superior vena cava (SVC) coil electrode 438. Typically, the right ventricular lead 430 is transvenously inserted into the heart so as to place the RV coil electrode 436 in the right ventricular apex, and the SVC coil electrode 438 in the superior vena cava. Accordingly, the right ventricular lead is capable of receiving cardiac signals, and delivering stimulation in the form of pacing and shock therapy to the right ventricle.

To sense left atrial and ventricular cardiac signals and to provide left chamber pacing therapy, pacer/ICD 10 is coupled to a “coronary sinus” lead 424 designed for placement in the “coronary sinus region” via the coronary sinus os for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus. Accordingly, an exemplary coronary sinus lead 424 is designed to receive atrial and ventricular cardiac signals and to deliver left ventricular pacing therapy using at least a left ventricular tip electrode 426, left atrial pacing therapy using at least a left atrial ring electrode 427, and shocking therapy using at least a left atrial coil electrode 428. With this configuration, biventricular pacing can be performed. Although only three leads are shown in FIG. 8, it should also be understood that additional stimulation leads (with one or more pacing, sensing and/or shocking electrodes) may be used in order to efficiently and effectively provide pacing stimulation to the left side of the heart or atrial cardioversion and/or defibrillation.

Although not shown, the leads may be provided with one or more pressure or PPG sensors, such as an LAP sensor installed adjacent the left atrium.

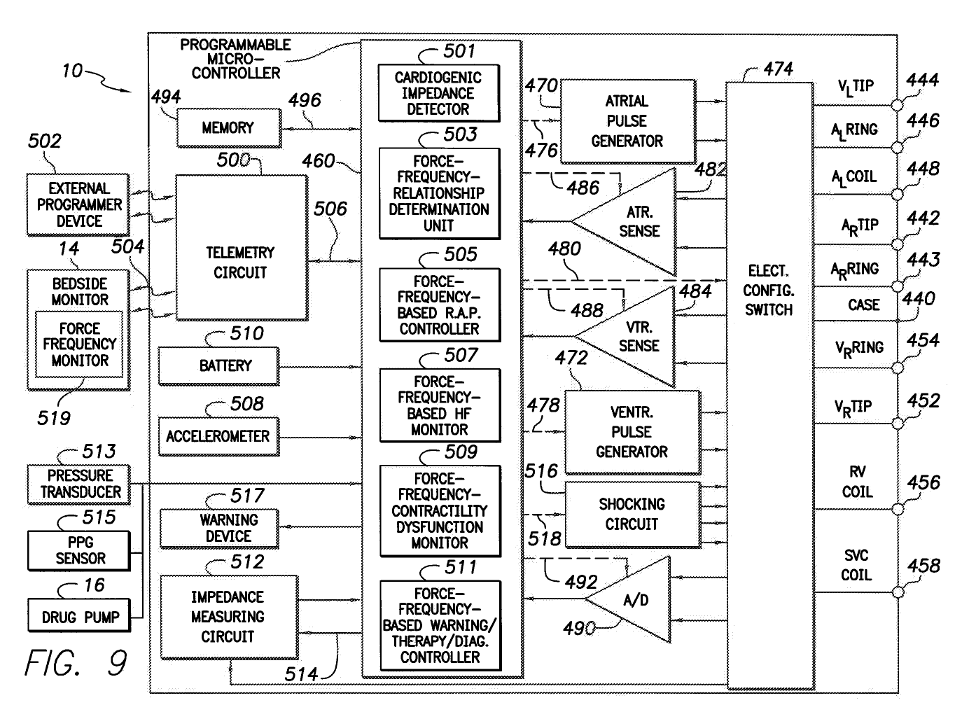

A simplified block diagram of internal components of pacer/ICD 10 is shown in FIG. 9. While a particular pacer/ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation as well as providing for the aforementioned diastolic function monitoring functions.

The housing 440 for pacer/ICD 10, shown schematically in FIG. 9, is often referred to as the “can”, “case” or “case electrode” and may be programmably selected to act as the return electrode for all “unipolar” modes. The housing 440 may further be used as a return electrode alone or in combination with one or more of the coil electrodes, 428, 436, 438, for shocking purposes. The housing 440 further includes a connector (not shown) having a plurality of terminals, 442, 443, 444,446, 448, 452, 454, 456, 458 (shown schematically and, for convenience, the names of the electrodes to which they are connected are shown next to the terminals). As such, to achieve right atrial sensing and pacing, the connector includes at least a right atrial tip terminal (AR TIP) 442 adapted for connection to the atrial tip electrode 422 and a right atrial ring (A_{R} RING) electrode 443 adapted for connection to right atrial ring electrode 423. To achieve left chamber sensing, pacing and shocking, the connector includes at least a left ventricular tip terminal (V_{L} TIP) 444, a left atrial ring terminal (A_{L} RING) 446, and a left atrial shocking terminal (A_{L} COIL) 448, which are adapted for connection to the left ventricular ring electrode 426, the left atrial tip electrode 427, and the left atrial coil electrode 428, respectively. To support right chamber sensing, pacing and shocking, the connector further includes a right ventricular tip terminal (V_{R} TIP) 452, a right ventricular ring terminal (V_{R} RING) 454, a right ventricular shocking terminal (Rᵥ COIL) 456, and an SVC shocking terminal (SVC COIL) 458, which are adapted for connection to the right ventricular tip electrode 432, right ventricular ring electrode 434, the RV coil electrode 436, and the SVC coil electrode 438, respectively.

At the core of pacer/ICD 10 is a programmable microcontroller 460, which controls the various modes of stimulation therapy. As is well known in the art, the microcontroller 460 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 460 includes the ability to process or monitor input signals (data) as controlled by a program code stored in a designated block of memory. The details of the design and operation of the microcontroller 460 are not critical to the invention. Rather, any suitable microcontroller 460 may be used that carries out the functions described herein. The use of microprocessor-based control circuits for performing timing and data analysis functions are well known in the art.

As shown in FIG. 9, an atrial pulse generator 470 and a ventricular/impedance pulse generator 472 generate pacing stimulation pulses for delivery by the right atrial lead 420, the right ventricular lead 430, and/or the coronary sinus lead 424 via an electrode configuration switch 474. It is understood that in order to provide stimulation therapy in each of the four chambers of the heart, the atrial and ventricular pulse generators 470, 472, may include dedicated, independent pulse generators, multiplexed pulse generators or shared pulse generators. The pulse generators 470, 472, are controlled by the microcontroller 460 via appropriate control signals 476, 478, respectively, to trigger or inhibit the stimulation pulses.

The microcontroller 460 further includes timing control circuitry (not separately shown) used to control the timing of such stimulation pulses (e.g., pacing rate, atrio-ventricular (AV) delay, atrial interconduction (A-A) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 474 includes a plurality of switches for connecting the desired electrodes to the appropriate I/O circuits, thereby providing complete electrode programmability. Accordingly, the switch 474, in response to a control signal 480 from the microcontroller 460, determines the polarity of the stimulation pulses (e.g., unipolar, bipolar, combipolar, etc.) by selectively closing the appropriate combination of switches (not shown) as is known in the art.

Atrial sensing circuits 482 and ventricular sensing circuits 484 may also be selectively coupled to the right atrial lead 420, coronary sinus lead 424, and the right ventricular lead 430, through the switch 474 for detecting the presence of cardiac activity in each of the four chambers of the heart. Accordingly, the atrial and ventricular sensing circuits 482, 484, may include dedicated sense amplifiers, multiplexed amplifiers or shared amplifiers. The switch 474 determines the “sensing polarity” of the cardiac signal by selectively closing the appropriate switches, as is also known in the art. In this way, the clinician may program the sensing polarity independent of the stimulation polarity. Each sensing circuit 482, 484, preferably employs one or more low power, precision amplifiers with programmable gain and/or automatic gain control, bandpass filtering, and a threshold detection circuit, as known in the art, to selectively sense the cardiac signal of interest. The automatic gain control enables pacer/ICD 10 to deal effectively with the difficult problem of sensing the low amplitude signal characteristics of atrial or ventricular fibrillation. The outputs of the atrial and ventricular sensing circuits 482, 484, are connected to the microcontroller 460 which, in turn, are able to trigger or inhibit the atrial and ventricular pulse generators 470, 472, respectively, in a demand fashion in response to the absence or presence of cardiac activity in the appropriate chambers of the heart.

For arrhythmia detection, pacer/ICD 10 utilizes the atrial and ventricular sensing circuits 482, 484, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (e.g., P-waves, R-waves, and depolarization signals associated with fibrillation which are sometimes referred to as “F-waves” or “Fib-waves”) are then classified by the microcontroller 460 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (A/D) data acquisition system 490. The data acquisition system 490 is configured to acquire intracardiac electrogram signals, convert the raw analog data into a digital signal, and store the digital signals for later processing and/or telemetric transmission to an external device 502. The data acquisition system 490 is coupled to the right atrial lead 420, the coronary sinus lead 424, and the right ventricular lead 430 through the switch 474 to sample cardiac signals across any pair of desired electrodes. The microcontroller 460 is further coupled to a memory 494 by a suitable data/address bus 496, wherein the programmable operating parameters used by the microcontroller 460 are stored and modified, as required, in order to customize the operation of pacer/ICD 10 to suit the needs of a particular patient. Such operating parameters define, for example, the aforementioned thresholds as well as pacing pulse amplitude or magnitude, pulse duration, electrode polarity, rate, sensitivity, automatic features, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable pacer/ICD 10 may be non-invasively programmed into the memory 494 through a telemetry circuit 500 in telemetric communication with the external device 502, such as a programmer, transtelephonic transceiver or a diagnostic system analyzer. The telemetry circuit 500 is activated by the microcontroller by a control signal 506. The telemetry circuit 500 advantageously allows intracardiac electrograms and status information relating to the operation of pacer/ICD 10 (as contained in the microcontroller 460 or memory 494) to be sent to the external device 502 through an established communication link 504. Pacer/ICD 10 further includes an accelerometer or other physiologic sensor 508, commonly referred to as a “rate-responsive” sensor because it is typically used to adjust pacing stimulation rate according to the activity or exercise state of the patient. However, the physiological sensor 508 may further be used to detect changes in cardiac output, changes in the physiological condition of the heart, or diurnal changes in activity (e.g., detecting sleep and wake states) and to detect arousal from sleep. Accordingly, the microcontroller 460 responds by adjusting the various pacing parameters (such as rate, AV Delay, V-V Delay, etc.) at which the atrial and ventricular pulse generators 470, 472, generate stimulation pulses. While shown as being included within pacer/ICD 10, it is to be understood that the physiologic sensor 508 may also be external to pacer/ICD 10, yet still be implanted within or carried by the patient. A common type of rate responsive sensor is an activity sensor incorporating an accelerometer or a piezoelectric crystal, which is mounted within the housing 440 of pacer/ICD 10. Other types of physiologic sensors are also known, for example, sensors that sense the oxygen content of blood, respiration rate and/or minute ventilation, pH of blood, ventricular gradient, etc.

The pacer/ICD additionally includes a battery 510, which provides operating power to all of the circuits shown in FIG. 9. The battery 510 may vary depending on the capabilities of pacer/ICD 10. If the system only provides low voltage therapy, a lithium iodine or lithium copper fluoride cell may be utilized. For pacer/ICD 10, which employs shocking therapy, the battery 510 must be capable of operating at low current drains for long periods, and then be capable of providing high-current pulses (for capacitor charging) when the patient requires a shock pulse. The battery 510 should also have a predictable discharge characteristic so that elective replacement time can be detected. Accordingly, pacer/ICD 10 is preferably capable of high voltage therapy and appropriate batteries.

As further shown in FIG. 9, pacer/ICD 10 is shown as having an impedance measuring circuit 512 which is enabled by the microcontroller 460 via a control signal 514. Uses for an impedance measuring circuit include, but are not limited to, detecting signals from which cardiogenic impedance can be derived, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves, etc. The impedance measuring circuit 512 is advantageously coupled to the switch 474 so that any desired electrode may be used.

In the case where pacer/ICD 10 is intended to operate as an implantable cardioverter/defibrillator (ICD) device, it detects the occurrence of an arrhythmia, and automatically applies an appropriate electrical shock therapy to the heart aimed at terminating the detected arrhythmia. To this end, the microcontroller 460 further controls a shocking circuit 516 by way of a control signal 518. The shocking circuit 516 generates shocking pulses of low (up to 0.5 joules), moderate (0.5-10 joules) or high energy (11 to 40 joules), as controlled by the microcontroller 460. Such shocking pulses are applied to the heart of the patient through at least two shocking electrodes, and as shown in this embodiment, selected from the left atrial coil electrode 428, the RV coil electrode 436, and/or the SVC coil electrode 438. The housing 440 may act as an active electrode in combination with the RV electrode 436, or as part of a split electrical vector using the SVC coil electrode 438 or the left atrial coil electrode 428 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 5-40 joules), delivered asynchronously (since R-waves may be too disorganized), and pertaining exclusively to the treatment of fibrillation. Accordingly, the microcontroller 460 is capable of controlling the synchronous or asynchronous delivery of the shocking pulses.

Microcontroller 460 also includes various components directed to determining and exploiting the force-frequency relationship. In particular, the microcontroller includes a cardiogenic impedance detector 501 operative to derive cardiogenic impedance signals from the impedance signals detected by the impedance measuring circuit 512. A force-frequency relationship determination unit 503 is operative to determine the force-frequency relationship for the patient based on surrogate contractile force parameters derived from cardiogenic impedance signals or from pressure signals received from a pressure sensor 513 and/or a PPG sensor 515. For clarity, these two sensors are shown in block diagram form with direct connections to the microcontroller. It should be understood, however, that appropriate electrodes may need to be provided on the device housing to receive signals from these devices.

The microcontroller also includes a force-frequency-based rate-adaptive pacing (RAP) controller 505 operative to control rate-adaptive pacing based on changes in the force-frequency relationship. In this example, a force-frequency-based heart failure monitor 507 is also provided, which is operative to detect an indication of heart failure based on changes in the force-frequency relationship. In the example, a force-frequency-based contractility dysfunction monitor 509 is also provide, which is operative to detect an indication of contractility dysfunction based on changes in the force-frequency relationship. Still further, a force-frequency-based warning/therapy/diagnostics controller 511 is provided. In implementations where a drug pump 16 is included, controller 511 controls the delivery of medications via the drug pump. Diagnostic data is stored within memory 494. Warning signals may be relayed to the patient via internal warning device 517 or via bedside monitor 14 or programmer 502.

Depending upon the implementation, the various components of the microcontroller may be implemented as separate software modules or the modules may be combined to permit a single module to perform multiple functions. In addition, although shown as being components of the microcontroller, some or all of these components may be implemented separately from the microcontroller.

When used in conjunction with an external system, the external system can perform some of the force-frequency monitoring functions, such as by determining the slope and/or abscissa values of the force-frequency curve based on data transmitted from the pacer/ICD. This is shown by way of force-frequency monitor 519 installed within the bedside monitor. In other words, not all of the functions need be performed by the pacer/ICD but functions can be distributed among various systems, some implanted within the patient, others external.

What have been described are various systems and methods for use with a pacer/ICD or an external system used in conjunction with a pacer/ICD. However, principles of the invention may be exploiting using other implantable medical systems. Thus, while the invention has been described with reference to particular exemplary embodiments, modifications can be made thereto without departing from the scope of the invention.

What is claimed is:

1. A method for use with an implantable medical device for implant within a patient, wherein the device is equipped to perform rate-adaptive pacing, the method comprising:
determining a force-frequency relationship for the heart of the patient, representative of the relationship between cardiac stimulation frequency and contractile force; and
controlling rate-adaptive pacing based on the force-frequency relationship.

2. The method of claim 1 further including detecting an indication of a lack of responsiveness to rate-adaptive pacing within the patient based on the force-frequency relationship.

3. The method of claim 2 wherein controlling rate-adaptive pacing based on the force-frequency relationship includes deactivating rate-adaptive pacing in response to the indication of a lack of responsiveness to rate-adaptive pacing.

4. The method of claim 2 wherein controlling rate-adaptive pacing based on the force-frequency relationship includes lowering a maximum rate-adaptive pacing rate in response to the indication of a lack of responsiveness to rate-adaptive pacing.

5. The method of claim 2 wherein controlling rate-adaptive pacing based on the force-frequency relationship includes decreasing a reaction time associated with rate-adaptive pacing in response to the indication of a lack of responsiveness to rate-adaptive pacing.

6. The method of claim 2 wherein detecting an indication of a lack of responsiveness to rate-adaptive pacing within the patient based on the force-frequency relationship includes determining at least one of a slope and an abscissa parameter representative of the force-frequency relationship and then determining whether the parameter is below a threshold indicative of lack of responsiveness to rate-adaptive pacing.

7. The method of claim 2 further including detecting an activity level of the patient and wherein controlling rate-adaptive pacing based on the force-frequency relationship is performed only while the activity level of the patient is above a threshold indicative of patient exercise.

8. The method of claim 1 wherein the device is equipped to pace the heart at differing pacing rates and wherein determining a force-frequency relationship for the heart of the patient includes:
detecting values representative of contractile force within the heart of the patient at various heart rates; and
recording the values representative of contractile force along with values representative of heart rate as the force-frequency relationship.

9. The method of claim 8 wherein detecting values representative of contractile force at various heart rates includes actively changing a pacing rate.

10. The method of claim 9 wherein actively changing the pacing rate includes activating rate adaptive pacing in response to patient activity and detecting the values representative of contractile force at various heart rates during rate adaptive pacing.

11. The method of claim 8 wherein detecting values representative of contractile force at various heart rates includes passively allowing the heart rate to change.

12. The method of claim 1 wherein determining the force-frequency relationship includes determining one or both of a slope of the force-frequency relationship and an abscissa of the force-frequency relationship.

13. The method of claim 1 wherein detecting values representative of contractile force includes detecting one or more of: a maximum time rate of change of cardiogenic impedance (dZ/dt); a time rate of change of cardiac pressure (dP/dt); a peak systolic pressure (P); and a maximum peak-to-peak amplitude of cardiogenic impedance (Z).

14. The method of claim 13 wherein detecting values representative of a time rate of change of cardiogenic impedance dZ/dt includes detecting cardiogenic impedance over time using one or more pacing/sensing electrodes.

15. The method of claim 13 wherein detecting values representative of a time rate of change of cardiac pressure (dP/dt) includes detecting left atrial pressure (LAP) using a pressure sensor.

16. The method of claim 13 wherein detecting values representative of peak systolic pressure includes detecting systolic pressure using a pressure sensor and then detecting its peak magnitude within at least one cardiac cycle.

17. The method of claim 1 further including detecting an indication of heart failure within the patient based on the force-frequency relationship.

18. The method of claim 1 further including detecting an indication of contractility dysfunction within the patient based on the force-frequency relationship.

19. The method of claim 1 further including controlling at least one device function based on the force-frequency relationship.

20. The method of claim 19 wherein controlling at least one device function based on the force-frequency relationship includes controlling delivery of pharmaceutical therapy.

21. The method of claim 20 wherein controlling delivery of pharmaceutical therapy includes initiating delivery of compounds intended to reduce ventricular-vascular stiffing in response to a decrease in at least one of a slope and an abscissa parameter representative of the force-frequency relationship.

22. The method of claim 19 wherein controlling at least one device function includes recording diagnostic data representative of the force-frequency relationship.

23. The method of claim 19 wherein controlling at least one device function includes generating warning signals in response to a significant change in the force-frequency relationship.

24. A system for use with an implantable medical device for implant within a patient, wherein the device is equipped to perform rate-adaptive pacing, the system comprising:
a force-frequency relationship determination unit operative to determine a force-frequency relationship for the heart of the patient, representative of the relationship between cardiac stimulation frequency and contractile force; and
a force-frequency-based rate-adaptive pacing controller operative to control rate-adaptive pacing based on the force-frequency relationship.

26. The system of claim 24 further including a force-frequency-based contractility dysfunction monitor.

25. The system of claim 24 further including a force-frequency-based heart failure monitor.

27. The system of claim 24 further including a force-frequency-based therapy controller.

29. The system of claim 24 further including a force-frequency-based warning controller operative to generate warning signals based on the force-frequency relationship.

30. A system for use with an implantable medical device for implant within a patient, wherein the device is equipped to perform rate-adaptive pacing, the system comprising:
means for determining a force-frequency relationship for the heart of the patient, representative of the relationship between cardiac stimulation frequency and contractile force; and
means for controlling rate-adaptive pacing based on the force-frequency relationship.
